(19) United States
(12) Patent Application Publication
(10) Pub. No.: US 2013/0345290 A1
(43) Pub. Date: Dec. 26, 2013

(54) PHARMACEUTICAL COMPOSITION CONTAINING L-DNA

(71) Applicant: Volker A. Erdmann, Berlin (DE)

(72) Inventor: Volker A. Erdmann, Berlin (DE)

(21) Appl. No.: 13/977,088

(22) PCT Filed: Jan. 2, 2012
(86) PCT No.: PCT/DE2012/000008

§ 371 Date: Sep. 4, 2013

(30) Foreign Application Priority Data
Dec. 31, 2010 (DE) 10 2010 056 610.1

(51) Int. Cl. C12N 15/113; C12N 15/11

(52) U.S. Cl. CPC C12N 15/113; C12N 15/11
USPC 514/44 A; 536/23.1; 530/391.1; 514/44 R; 536/24.5

(57) ABSTRACT

The invention relates to the use of an L-DNA which is capable of binding to an L-RNA, in particular in an antisense reaction, and optionally of cleaving the L-RNA in the range of a target sequence of the L-RNA, for preparing a pharmaceutical composition for the treatment of undesired physiological side reactions due to the administration of a therapeutic molecule containing the L-RNA. The L-DNA can alternatively also be used for cleaving an endogenous target RNA or DNA.

### FIELD OF THE INVENTION

The invention relates to a pharmaceutical composition comprising an L-DNA, to the use of an L-DNA for preparing a pharmaceutical composition, and to a method for preparing such a pharmaceutical composition.

### BACKGROUND OF THE INVENTION AND PRIOR ART

Aptamers are in most cases double-stranded D-nucleic acids, which bind specifically to an arbitrary target molecule, in an analogous manner to an antibody/antigen reaction (Ellington, A. D. et al., Nature 346:818-822 (1990)). For a given target molecule, specific aptamers are isolated, for example by the SELEX method, from nucleic acid libraries (Tuerk, C. et al., Science 249:505-510 (1990)).

In the therapeutic sector, it is the purpose of aptamers, inter alia, to bind undesired metabolites and thereby inhibit them. Just as an example, oncogenic gene products are mentioned here. For the therapeutic use of aptamers, it is disadvantageous that they have an unfavorable pharmacokinetics, i.e. they will very quickly be degraded, for example by endogenous nucleases. Independently of this, aptamers are anyway relatively small molecules, which are therefore discharged relatively quickly through the kidney.

Spiegelmers are basically aptamers, but differ from them in that they are formed from L-nucleotides. Spiegelmers may be single or double-stranded. Through the use of L-nucleotides, decomposition by endogenous nucleases is prevented, and thus the pharmacokinetics is significantly improved, i.e. the residence time in the serum is extended. For instance, in the document Boisgard, R. et al., Eur Journal of Nuclear Medicine and Molecular Imaging 32:470-477 (2005), it is described that non-functional Spiegelmers are metabolically completely stable even over a period of 2 hours. In this document, the diagnostic use of Spiegelmers is also described, the Spiegelmer being coupled with a, for example, radioactive reporter substance.

Identifying Spiegelmers being specific for a given target molecule can be made for example as described in the document Klussmann, S. et al., Nat Biotechnol 14:1112-1115 (1996). With regard to Spiegelmers and their therapeutic applications, reference is also made to the document Vater, A. et al., Curr Opin Drug Discov Devel 6:253-261 (2003).

In the therapeutic use of Spiegelmers, it has hitherto been assumed that Spiegelmers are not immunogenic (Wlotzka et al., Proc Natl Acad Sci USA 99:8898-8902 (2002)). Investigations, which are presented in the present specification, show, however, that L-nucleic acids in an organism are not necessarily entirely free from side effects. It follows that when using Spiegelmers, there is a not negligible risk of an undesired physiological side reaction, such as an immune response and/or an undesired enzymatic reaction with endogenous RNA (including a regulatory RNA), or also an antisense inhibition (Watson-Crick reaction) of an endogenous nucleic acid, when administering to a patient. Especially in the light of the adverse experiences with the monoclonal antibody TGN1412 encountered in the clinical phase 1 and taking into account that the residence time of Spiegelmers, due to the aforementioned conditions, is comparatively very high, it would be desirable to have an antidote for a Spiegelmer ready to be used when administering the Spiegelmer, so that when an undesired physiological side reaction occurs, immediately the antidote can be administered, and thus the (biologically active) Spiegelmer level in the serum can quickly be reduced.

From other contexts, namely the ribozyme-catalyzed stereoselective Diels-Alder reaction, L-ribozymes are known, reference being made to the documents Seelig, B. et al., Angew. Chem. Int., 39:4576-4579 (2000) and Seelig, B. et al., Angew. Chem 112:4764-4768 (2000).

Furthermore, various approaches for the inhibition of endogenous nucleic acids, for example mRNA or other non-coding nucleic acids are known. These include, for example, but not exclusively antisense nucleic acids, siRNA, miRNA, piRNA, aptamers etc. By inhibition of such endogenous nucleic acids, metabolic processes can be controlled, inhibited or deflected, which is relevant in conjunction with tumor-associated RNA molecules, but in other medical fields, too. As an example of a tumor-associated gene, the H19 gene is mentioned here. As an example of a non-tumor-associated gene, the gene coding for phospholamban is mentioned here, which plays an important role in the context of heart failure.

From the document WO 2010/088899 A2, it is known to use L-ribozymes for cleaving Spiegelzymes (as an antidote) and/or endogenous RNA molecules.

L-DNA is per se known, for example from the document G. Hayashi et al., Nucleic Acids Symp Ser 49 (1):261-262 (2005), in which such nucleic acids are described as molecular tags.

### TECHNICAL OBJECT OF THE INVENTION

It is the technical object of the invention to provide improved means for cutting Spiegelmers and endogenous nucleic acids.

### BASICS OF THE INVENTION

For achieving this technical object, the invention teaches the use of an L-DNA for preparing a pharmaceutical composition, wherein the L-DNA is preferably capable of binding to an L-RNA, in particular an antisense reaction (inhibitory Watson-Crick reaction), and is optionally capable of cleaving the L-RNA in the range of a target sequence of the L-RNA, in particular for preparing a pharmaceutical composition for the treatment of undesired physiological side reactions, in particular immune reactions and/or undesired enzymatic or antisense reactions of the L-RNA with endogenous RNA (including a regulatory RNA), due to the administration of a therapeutic molecule comprising the L-RNA. Further, the use of an L-DNA for preparing a pharmaceutical composition for the treatment or prophylaxis of diseases accompanied by an overexpression of at least one endogenous gene, wherein the L-DNA is capable of binding to a target sequence of an endogenous D-DNA or target D-RNA coding for the gene, for example in an antisense reaction, and is optionally capable of cleaving said target sequence.

First of all, the invention is based on the finding that Spiegelmers, in contrast to previous assumptions, are not necessarily free from side reactions, but may rather be capable of cutting nucleic acids naturally occurring in an organism and of thus producing unpredictable side effects. Similarly, undesired antisense reactions, i.e. inhibition of an endogenous nucleic acid by Watson-Crick base bonds between the Spiegelmer and the endogenous nucleic acid is possible, regardless of enzymatic reactions of the bound Spiegelmer.

The invention is based on the further finding that L-DNA is surprisingly capable of cutting endogenous D-nucleic acids, RNA, DNA, or of binding thereto. This cannot automatically be expected. In addition, L-DNAs are particularly stable against enzymatic degradation, so that no (usually bulky) protection groups have to be attached at the molecules, thereby on the one hand advantageous pharmacokinetic properties being obtained, and on the other hand the reception in cells being enhanced.

A surprising advantage of L-DNA over L-RNA is that the activity of L-DNA in cells is higher compared to L-RNA, and reference is made to the embodiments.

The invention is based on these findings and on the technical teaching to provide L-DNA, i.e. L-DNAzymes that specifically cut an administered Spiegelmer or bind thereto in an inhibiting manner and thus destroy the physiological activity thereof, in particular in view of adverse side reactions. Examples of Spiegelmers are: Spiegelmer, NOXC89, NOXA42, NOXA50, NOXB11, NOXAl2, NOXE36, NOXF37 (all from NOXXON AG), Spiegelmers made by Eli Lilly & Co., NU172 of the company ARCA biopharm Inc., ARCHEMIX, ARC 1905, ARC 1779, ARC 183, ARC184, E10030, NU172, REG2, REG1 (all from Archemix Corp.), AS1411, AS1405 (both from Antisoma Research Ltd.), DsiRNA from Dicerna Pharmaceuticals Inc., RNA aptamer BEXCORE from BexCore Inc., ELAN from Elan Corp. Plc., or Macugen. By administration of such an L-DNA in pursuit of observing an undesired side reaction during the administration of a Spiegelmer, consequently the cause of the undesired side reaction can quickly, effectively and highly selectively be removed from the metabolism, and that again with an extremely low risk of side effects of the administration of L-DNA. The latter is based not only on the structure of the L-DNA from L-deoxyribonucleotides, but additionally on the high selectivity of L-DNA, namely directed to the target sequence of the Spiegelmer. As a result, a highly effective and highly selective antidote against a therapeutically employed Spiegelmer is obtained, and undesired side reactions of the Spiegelmer can be attacked effectively, quickly and freely from side effects.

Basically a specific L-DNA can be constructed against each RNA molecule, including aptamers, whether it is composed of D or L-nucleotides, that specific L-DNA cutting a target sequence of the RNA molecule and thus cleaving it (acting as a ribozyme) or binding thereto in an inhibiting manner (antisense reaction). An essential characteristic of such an L-DNA is thus the sequence-specific binding to the target sequence. This also means, however, that for any given target sequence, a partial sequence of an L-DNA can be created by that the partial sequence of the L-DNA containing a cleavage site, for example, hybridizes with the target sequence. Therefore, it is not appropriate in the present invention, to structurally specify only certain L-DNA partial sequences with respect to specific target sequences. The target sequences and L-DNA partial sequences given in the examples are therefore exemplary only, and the person skilled in the art can readily determine for each target sequence of a Spiegelmer the matching, namely hybridizing L-DNA partial sequence and synthesize the L-DNA based on the information about the L-DNA sequence with conventional technical means.

In general, the therapeutic molecule may be a Spiegelmer, or the L-RNA may be covalently bonded to an aptamer. The therapeutic molecule may however also comprise an L-DNA (in addition to an aptamer, for example) or consist thereof. A combination Spiegelmer/aptamer may exist, for example in the case of an aptamer stabilized against nucleases. Then, the therapeutic benefit of the invention is that by cutting the L-RNA or L-DNA, the aptamer is made accessible for nucleases, whereby eventually an aptamer possibly causing side effects can be eliminated from the serum in a comparatively short time.

It is however also possible that the L-DNA is covalently bonded to an aptamer or an antibody. In this case, the aptamer or the antibody may be selected, for example, such that due to the interaction of the aptamer or of the antibody with the cell surfaces, the entire construct of L-DNA and aptamer or antibody is introduced into the cell.

A suitable L-DNA may be directed against a conserved cleavage site in the substrate sequence and itself comprise conserved nucleotides, as shown in FIG. 1 and in particular FIG. 1a. A specific example thereof is also shown in FIG. 1c.

The pharmaceutical composition contains the L-DNA in at least the dose corresponding to the dose of administration of the L-RNA, preferably in a dose that is 2 to 10 times, referred to the number of molecules or moles, the dose of administration of the L-RNA. An overdose, compared to the dose of the L-RNA, is recommended to make sure that all L-RNA to be eliminated is reacted. The absolute doses provided according to the invention will strictly be determined in the given relative proportions according to the specified doses of the L-RNA and can therefore easily be determined and established by the man skilled in the art having knowledge of the prescribed doses for the L-RNA.

In a preferred embodiment of the invention, the pharmaceutical composition additionally contains a nucleic acid, in particular a 5 to 100-mer, preferably a 5 to 25-mer, which is capable of melting a double-stranded L-RNA in the range of the target sequence thereof. These are sequences, which hybridize with partial sequences that are adjacent to the target sequence. Thereby it is achieved that areas of the L-RNA to be cleaved, which are generally not accessible for steric reasons, due to the tertiary structure of L-RNA, are made accessible to the L-DNA. Further, it is achieved that the desired cutting sites are very specifically cleaved, not however corresponding doublets or triplets with other neighboring sequences.

The invention further relates to a pharmaceutical composition comprising an L-DNA for the treatment of undesired physiological side reactions, in particular immune reactions, due to the administration of a therapeutic molecule comprising the L-RNA.

According to the invention, L-DNA may however also be used for cleaving (endogenous or exogenous, for example derived from viral or bacterial sources in pursuit of an infection) nucleic acids, substantially RNA, but also DNA, or for the inhibition thereof by an antisense reaction at the endogenous nucleic acids. With regard to cutting DNA, reference is made to the documents Lu, Y., et al., Current Opinions in Biotechnology 17:580-588 (2006), and Jiang, D., et al., FEBS 277 (11):2543-2549 (2010). In particular, those diseases can be treated thereby that are accompanied by a specific RNA or DNA, or by the overexpression of an expression product coded thereby. Consequently, the L-DNA will act as an inhibitor with regard to this expression product, namely by that the expression is inhibited or reduced by cleavage of the RNA or DNA coding therefor or by antisense binding thereto. The specific target sequence (i.e. of the RNA or DNA to be cleaved or bound) is in so far irrelevant for the purpose of the invention, as any targets can be inhibited thereby. It is only necessary to adjust the L-DNA or the sequence thereof to the sequence of the target sequence in the region of the selected cleavage site in the manner described above. This allows, in principle, to include all indications, provided the disease to be treated is causally related to the corresponding target sequence. In the following, just examples are given that however do not limit the applicability of the invention in any way.

Regarding the pharmaceutical composition, all of the above and below statements apply in an analogous manner.

Finally, the invention relates to a method for preparing such a pharmaceutical composition, wherein a sequence is prepared and synthesized from L-deoxyribonucleotides, which is capable of binding to a predetermined sequence of L-ribonucleotides, or to a predetermined sequence of D-ribonucleotides or D-deoxyribonucleotides, in particular capable of an antisense reaction, and optionally of cleaving said sequence, the L-DNA thus obtained being prepared in a pharmacologically effective dose for administration. Typically, the L-DNA is mixed with galenic auxiliary and/or carrier substances. For the preparation, it is also possible to covalently couple conventional substances, which promote the endocytosis (of the L-DNA), to the L-DNA or admix them separately into the composition.

Basically, one or more physiologically acceptable auxiliary and/or carrier substances may be mixed with the L-DNA, and the mixture is galenically prepared for local or systemic administration, in particular orally, parenterally, for infusion into a target organ, for injection (for example iv, im, intracapsular or intralumbar administration), for the application in the periodontal pockets (space between the root of the tooth and gum) and/or for inhalation. The choice of additives and/or auxiliary substances will depend on the selected dosage form. The galenic preparation of the pharmaceutical composition according to the invention may be made in a conventional way. As counterions for ionic compounds for example, Mg⁺⁺, Pb⁺⁺, Mn⁺⁺, Ca⁺⁺, CaCl⁺, Na⁺, K⁺, Li⁺ or cyclohexylammonium, and Cl⁻, Br⁻, acetate, trifluoroacetate, propionate, lactate, oxalate, malonate, maleinate, citrate, benzoate, salicylate, putrescine, cadaverine, spermidine, spermine, etc. may be used. Suitable solid or liquid pharmaceutical preparation forms are, for example, granules, powders, pills, tablets, (micro) capsules, suppositories, syrups, elixirs, suspensions, emulsions, drops or solutions for injection (iv, ip, im, sc) or nebulization (aerosols), preparation forms for dry powder inhalation, transdermal systems, as well preparations with protracted release of active ingredient, for the preparation of which conventional auxiliaries such as carrier substances, disintegrants, binders, coating agents, swelling agents, glide agents or lubricants, flavorings, sweeteners and solubilizers are used. It is also possible to encapsulate the active substance in preferably biodegradable nanocapsules or to incorporate it in a biodegradable or stable manner in pores of porous nanoobjects, for example for the preparation of a composition for inhalation. Auxiliary substances may be, for example, sodium carbonates, magnesium carbonate, magnesium bicarbonate, titanium dioxide, lactose, mannitol and other sugars, talc, milk protein, gelatin, starch, cellulose and its derivatives, animal and vegetable oils such as cod liver oil, sunflower oil, peanut oil or sesame oil, polyethylene glycols and solvents, such as sterile water and monovalent or polyvalent alcohols, for example glycerol. A pharmaceutical composition according to the invention can be prepared by that at least one substance used according to the invention is mixed in a defined dose with a pharmaceutically suitable and physiologically acceptable carrier and if applicable other suitable active compounds, additives or auxiliary substances and prepared to obtain the desired form of administration. Suitable diluents are polygly-cols, water, and buffer solutions. Suitable buffer substances are for example N,N′-dibenzylethylenediamine, diethanolamine, ethylenediamine, N-methylglucamine, N-benzylphenethylamine, diethylamine, phosphate, sodium bicarbonate, or sodium carbonate. However, the process can also be performed without a diluent. Physiologically acceptable salts are salts with inorganic or organic acids such as lactic acid, hydrochloric acid, sulfuric acid, acetic acid, citric acid, p-toluenesulfonic acid, or with inorganic or organic bases, such as NaOH, KOH, Mg(OH)₂, diethanolamine, ethylenediamine, or with amino acids such as arginine, lysine, glutamic acid, etc., or with inorganic salts such as CaCl₂, NaCl, or the free ions thereof such as Ca⁺⁺, Na⁺, Pb⁺⁺, Cl⁻, SO₄⁻ or corresponding salts and free ions of Mg⁺⁺ or Mn⁺⁺, or combinations thereof. They are prepared by standard methods. Preferably, a pH in the range between 5 and 9, in particular between 6 and 8, is used.

Of an independent relevance is the variant of the invention already mentioned above, which comprises the use of an L-DNA for preparing a pharmaceutical composition for the treatment or prophylaxis of diseases, which are accompanied by an overexpression of at least one endogenous gene, wherein the L-DNA is capable of binding to a target sequence of an endogenous target D-RNA or target D-DNA coding for the gene, in particular for an antisense reaction, and is optionally capable of cleaving said target sequence. A treatment or prophylaxis of viral or bacterial infections is possible, when the L-DNA is adapted for binding to a target sequence of the respective virus or bacterium. Other than that, the above explanations apply in an analogous manner. In this context, another variation of the above aspect of the invention is important, using an L-DNA for preparing a pharmaceutical composition for the treatment or prophylaxis of diseases, which are accompanied by an infection of a mammal with a microorganism, wherein the L-DNA is capable of cleaving a (or of binding, in particular by an antisense reaction, to a) target sequence of a target D-RNA, or target D-DNA coding for a gene of the microorganism. Those microorganisms may for example be viruses, bacteria and fungi. In general, the L-DNA can be used for binding to or for cleaving nucleic acids of any microorganism with at least partly known genetic sequences, those portions of the genetic sequences being selected for cleaving that for example attenuate or inhibit the activity of the microorganism and/or its capability of replication and/or attenuate or inhibit the binding to cell surfaces.

This variant is based on that the L-DNA can also be used for inhibiting by an antisense reaction with or without cleavage of D-RNA, particularly mRNA or regulatory RNA, such as, but not limited to siRNA, microRNA, shRNA, ncRNA, tRNA, rRNA, etc., but also for cleaving D-DNA or for binding thereto. As a result, genes or proteins encoded thereby can be inhibited. This is a therapeutic benefit for all diseases accompanied by the overexpression of certain genes, compared with the expression of the non-diseased organism.

This variant has the advantage on the one hand that the cleavage of the target sequence and the binding thereto occur with very high specificity, and therefore no other interference with the regulatory system takes place. In addition, side effects such as for example they occur with the use of D-inhibitory nucleic acids such as siRNA, are safely avoided.

Finally, it is possible to combine two L-DNA's, the sequences being selected such that double-stranded DNA or RNA can also be cut.

Within the scope of the use according to the invention of an L-DNA for preparing a pharmaceutical composition, further variants are possible.

Firstly, an L-DNA cannot be used for binding to another nucleic acid, but basically in a manner analogous to the known applications of aptamers from D-nucleic acids. This means that by means of an L-DNA according to the invention, in principle, any target molecule can be bound in an organism and thus inhibited. The whole technology of aptamers known to the man skilled in the art can accordingly be transferred to L-DNA aptamers.

An L-DNA binding to a given target is available in a subsequent process. A selected target molecule is bound at an immobile (or solid phase, for example also magnetic beads) phase of a screening assay. The screening assay comprises, in addition to the immobile phase, a mobile phase (generally an aqueous solution, in which nucleic acids and the target molecule are stable and can bind), which is in contact with the immobile phase or is brought into contact therewith. The mobile phase comprises an L-DNA library, i.e. polynucleotides, typically a length of 10 to 500 nucleotides, the sequences of which vary, are usually randomized. Such L-DNA libraries can be prepared by conventional synthesis methods known from the generation of D-DNA libraries. By contacting, those L-DNA molecules bind to the target molecule, which are capable, because of their sequence, of forming stable van der Waals bonds to the target molecule. The bond strengths typically render dissociation constants with values below 100 pmol, in most cases below 1 pmol. Thereafter, the mobile phase (comprising the non-binding or only weakly binding nucleic acids) is separated from the immobile phase, for example by one or more washing stages. Then the immobile phase is contacted with L-DNA molecules bound to target molecules with a D-DNA library. D-DNA hybridizing with the L-DNA bound to the target molecule binds to the L-DNA, thereby forming a complex of target molecule/L-DNA/D-DNA is formed. Unbound D-DNA is removed with the mobile phase. From the complex thus obtained, the D-DNA is then eluted again in a conventional way, i.e. converted into a mobile phase. The resulting D-DNA molecules can now if applicable be amplified (for example by PCR), and in any event be sequenced. From the thus obtained sequence of the D-DNA, the complementary L-DNA can be determined and synthesized. Alternatively, the L-DNA can be eluted from the target molecule and sequenced using a sequencing method explained elsewhere in this description. The method described in this section can in principle also be performed without an immobile phase, then the target molecule, rather than being bound to the immobile phase, is bound to a marker molecule. The separation of unbound L-DNA from the complex target/bound L-DNA is then carried out by conventional methods by binding the marker molecule and separating molecules not carrying this marker molecule. Other than that, this alternative works in a manner being quite analogous to the above description.

The result is an L-DNA molecular species or a mixture of such species, which bind with high affinity to the target molecule. These can then be used in a pharmaceutical composition according to the invention comprising arbitrary indications. The indication will then depend on which target molecule has been detected as causally connected with a disease, and is to be inhibited for the treatment or prophylaxis of the same.

In a corresponding manner, an L-RNA binding to a target can be isolated and determined. In this case, an L-RNA library is then used instead of an L-DNA library.

In another alternative method, L-DNA or L-RNA binding to an (arbitrary) target molecule can be isolated or identified and prepared. For this purpose, the L-nucleic acid library is provided, wherein optionally a coupling molecule or marker molecule is bound to the nucleic acids, for example, biotin at the 5′ end. The target molecule is bound to a solid phase, for example magnetic beads. The solid phase is then contacted with the nucleic acid library. In this case, those L-nucleic acids bind to the target molecule that have a high binding affinity thereto. The solid phase is subjected to one or more washing steps, whereby the non-binding L-nucleic acids are removed. Then the L-nucleic acids are in turn eluted, and thus separated from the target molecules in a conventional way. Finally, then a sequencing of the resulting L-nucleic acids is carried out, as described elsewhere in this description.

The sequencing of L-nucleic acids can be carried out, irrespective of other aspects of the invention, in different ways. In a first method for sequencing an L-RNA or L-DNA, the L-nucleic acid is bound to a solid phase. This can for example take place by that the nucleic acid contains a coupling or marker molecule, such as biotin (as described above). Then, the solid phase carries a molecule being complementary to the coupling molecule and binding the latter, for example avidin or streptavidin. The L-nucleic acid thus bound to the solid phase is then contacted with a D-DNA library. In this case, those D-DNA molecules of the library hybridize with the L-nucleic acid, which contain complementary sequences or consist thereof. Then, the solid phase is washed in one or more washing steps, unbound D-DNA being removed. Then, the bound D-DNA is released from the L-nucleic acid. Subsequently, an amplification can be performed, for example by PCR. Thereafter, the D-DNA is sequenced. From the D-DNA sequence thus obtained, the complementary sequence of the L-nucleic acid can then be determined.

Alternatively, an L-nucleic acid, particularly an L-RNA, can be sequenced in a sequencing process, as follows. The nucleic acid carries at one end, for example at the 5′ end, a coupling or marker molecule (as described above), such as biotin. The nucleic acid will be broken up into a “ladder”, i.e. by means of hydrolysis, fragments of different lengths of the nucleic acid are obtained, in an ideal case from 1 base to the number of bases of the complete nucleic acid. In the case of an L-RNA, this may occur in the alkaline range, pH typically >8, usually 8.5 to 10. For this purpose, a commercial hydrolysis buffer with KOH or sodium bicarbonate can be used. The thus obtained fragments, which also carry the coupling molecule, are then bound to a solid phase. For this purpose, the solid phase includes a molecule being complementary to the coupling molecule and binding the latter, such as avidin or streptavidin. The solid phase is then subjected to one or more washing steps, whereby nucleic acid fragments are removed, which do not carry the coupling molecule. As a result, the “ladder” of marked nucleic acid fragments is left over. These are then eluted from the solid phase and investigated by mass spectrometry, in which case on the basis of the masses found and their distribution, the original sequence of the L-nucleic acid can be determined. In the case of the L-DNA, a corresponding procedure is followed, only that there the “ladder” is generated by hydrolysis in the acidic range, i.e. pH <6, better <5.

Regardless of the explanations described herein, and representing an independent invention, L-DNA may also be used for non-pharmaceutical purposes. One application is the marking of objects or persons with L-DNA for security and/or authentication purposes and/or for the identification of a person. The L-DNA is applied to the object or the person, and the presence and/or the sequence thereof is checked using appropriate methods.

Those objects may, in principle, be all objects, the authenticity of which is to be verified, which are to be marked for theft protection purposes, or for which an assignment to an owner is desirable. To the first group belong the so-called security and/or value documents, such as passports, identity cards, driving licenses, motor vehicle documents, visas, other identity and/or access documents, such as access cards, member ID cards, banknotes, tickets, tax stamps, postal stamps, credit cards, or self-adhesive labels (for example for product protection). The second group includes objects, which represent a substantive value and are to be secured against theft, such as jewelry, watches, other valuables, technical equipment, vehicles, etc. With the use of an L-DNA, objects can also be individualized, namely by that an object is marked with an L-DNA, which comprises a sequence being characteristic for the object and uniquely for this object. If such a sequence is assigned to a person or an owner, an assignment of the object to the person or owner can be achieved by determination of the sequence of the L-DNA applied onto the object.

Marking persons may be desirable for example in the case of an attack. The assaulted person can then spray the attacker for example by means of a spray containing an L-DNA, whereby the person can be identified by detecting the L-DNA on the person or on the person's clothing. Further, automatic spray devices may also be provided for example for marking persons unauthorizedly entering premises or leaving them. A spray device that is coupled to an alarm system, is activated when a sensor of the alarm system detects the presence of a person within the reach of the spray device. Then the person is sprayed with the solution contained in the spray device, which in turn contains the L-DNA, and identification may then, as above, be performed.

Although the use of D-nucleic acids for such purposes is known in the art, these D-nucleic acids have the disadvantage that they can be removed by an unauthorized person, for example by means of nuclease. This is disturbing in particular in cases where an object is to be secured against theft, or where a person has been marked, as by the use of a nuclease the marking is destroyed and is thus removed.

The invention described herein in so far relates to a method for marking an object or a person, wherein the object or the person or clothing thereof is provided with an L-DNA, and wherein the L-DNA is fixed on or in this object, the person or clothing. It also relates to an object having an L-DNA fixed thereon or therein. It further relates to a method for identifying an object or a person, wherein the object or the person or the person's clothing is subjected to an analysis for the presence of an L-DNA, optionally in addition to the sequencing thereof

The term marking denotes an identification of an object or a person by applying a feature on or at that object or person, which previously was not on or at the object or the person. In any case, this feature has a predetermined structure and cannot get on the object or the person by other circumstances than (intentional) marking

Fixing can be made by all technologies known for marking by means of nucleic acids. In the simplest case, a solution, in particular an aqueous solution containing the L-DNA, is applied on the object or person or on the person's clothing, and is dried. Preferably, however, the L-DNA is contained in a preparation, which additionally comprises a dissolved or dispersed binder. Basic preparations are in principle all not yet cured liquid or pasty paint binder preparations, adhesive preparations or the like, provided the pH thereof is less than 9, better less than 8. Solvents may be, in addition to water, all solvents being usual in paint technology or adhesive technology. This also applies to the binders and conventional additives to be used. This preparation is applied on the object to be marked or on the person to be marked. The solution or preparation to be applied contains, per ml, preferably between 10̂3 and 10̂12, in particular between 10̂3 and 10̂9 molecules of the L-DNA. Preferably, at least 10%, preferably at least 50% of the L-DNA molecules have an identical nucleotide sequence.

In a preferred variant of this invention, the L-DNA carries at the 5′ or 3′ end a covalently bonded photoluminescent reporter molecule group, which is furthermore preferably selected such that the luminescence, in particular fluorescence, occurs upon excitation with UV radiation. Reporter molecule groups may be all photoluminescent molecular groups being used in biochemistry. If an object or a person marked according to the invention is illuminated with UV light, the marking is visible to the eye because of the luminescence excited thereby, or can be detected by an apparatus. The L-DNA may carry at the opposite end a marker group, for example biotin, in a covalently bonded manner. Then, upon a positive detection of a marking with an L-DNA, a removal and sequencing of the latter can take place by any of the methods described above. By the sequence, then an assignment to a person or business unit registered under the measured sequence can be performed, if applicable.

In a refinement of this invention, the L-DNA, may contain at least one invariant sequence block and/or a variable sequence block. The invariant block is then identical for all or at least one group of markings with the L-DNA, i.e., all markings contain a partial sequence with the sequence of this sequence block. The variable sequence block may then be individualizing. This is useful, if the detection by illumination for example with UV should in addition be dependent on the presence of an L-DNA with said sequence block. This is distinguished from a lighting effect, which may occur by any luminescent substances, irrespective of the presence of an L-DNA according to the invention.

In this refinement, it is further advantageous, if the L-DNA used according to the invention has the structure of a molecular beacon. This is a single-stranded nucleic acid sequence having a hairpin or stem-loop structure, wherein the ends forming the stem carry on the one hand a luminescent molecule and on the other hand, opposite thereto, a quencher, for example dabcyl. At least one of the ends is a (typically 5 to 20 base pairs long) invariant sequence (sequence block). In the unhybridized state of the L-DNA, the fluorescence is suppressed by Förster resonance energy transfer to the quencher; when irradiated with UV light, no effect is seen. Proof of the L-RNA is then carried out by that the marked area is first sprayed with a solution of a nucleic acid being complementary to an invariant sequence block of this L-DNA, in particular L-DNA. The L-DNA hybridizes with the invariant sequence block, and thereby the luminescent molecule and the quencher are separated from each other. If now the marking is irradiated with UV light, fluorescence will be visible or can be detected by an apparatus. An elution and sequencing of a possible variable block sequence is then carried out, as described above.

In a variant of the above preferred embodiment, the L-DNA of the marking is not a single strand, but one end (one end always means either 3′ or 5′) of a (longer) single strand carries a luminescent molecule. This end constitutes an invariant sequence block of a predetermined number of bases. Thereto is hybridized a complementary L-DNA, which carries a quencher at one end, and that with the proviso that the quencher is arranged sufficiently close to the luminescent molecule to suppress the luminescence. The length of this complementary L-DNA is by at least 2, better by at least 5 bases shorter than the length of the invariant sequence block. If now a solution with an L-DNA, which is complementary to the invariant sequence block and the length of which is by at least 2, better by at least 5 bases longer than that L-DNA carrying the quencher, the latter will displace the L-DNA having the quencher and hybridize with the invariant sequence block. If now an irradiation with UV is carried out, the luminescent molecule will light up, and the marking will become visible or detectable by an apparatus. Subsequently, again, elution and sequencing may be carried out, in order to detect a potential variable sequence.

The invention thus also includes a registration system comprising a database, wherein in the database variable sequence blocks of different L-DNA markings are detected and assigned to a person, firm or agency. By means of this registration system, a variable sequence block determined from a marking can easily be allocated to the assigned agency, firm or person.

In the following the invention will be further explained with reference to s and examples.

### BRIEF DESCRIPTION OF THE DRAWINGS

FIG. 1(a) shows the general structures of L-DNA according to the invention; FIG. (1b) shows the general structures of L-DNA structure and hammerhead ribozyme; FIG. 1(c) shows each with target sequence specificities and conserved structural elements as well as comparison of the binding of a specific L-DNA; and FIG. 1(d) shows each with target sequence specificities and conserved structural elements as well as a specific hammerhead ribozyme after binding to the same target sequence of the green fluorescent protein GFP (b), representation of the secondary structures (see also Zaborowska, Z., et al., The Journal of Biological Chemistry 277 (43):40617-40622 (2002), Kim, K., et al, Bull Korean Chem. Sco. 27 (5):657ff (2006), and Hertel, K. J., et al. Nucleic Acids Research 20 (12):3252ff (1992)),

FIG. 2: shows an analysis of the cleavage of an L and D-GFP target sequence by L and D-DNA hammerhead ribozyme,

FIG. 3: shows a comparison of the dependency of the cleavage of the D-GFP target sequence by L-DNA (L-Dz) and the L-GFP target sequence by D-DNA (D-Dz) on the MgCl₂ concentration,

FIG. 4: shows the dependency of the cleavage of the D-GFP target sequence by L-DNA (L-Dz) on the MgCl₂ concentration, with determination of cleavage site,

FIG. 5: shows the comparison of the activities of various DNAzymes and RNAzymes in GFP-transfected cells at different MgCl₂ concentrations, and 24 hours of incubation,

FIG. 6: shows a quantification of the results of FIG. 5 by specifying the fluorescence intensities,

FIG. 7: shows a direct comparison of the activities of L-DNAzyme and D-DNAzyme at different MgCl₂ concentrations, and 24 hours of incubation,

FIG. 8: shows a quantification of the results of FIG. 7 by specifying the fluorescence intensities,

FIG. 9: shows the subject matter of FIG. 5, but after 48 hours of incubation, and

FIG. 10: shows a quantification of the results of FIG. 9 by specifying the fluorescence intensities.

### DETAILED DESCRIPTION

### EXAMPLE 1

### Cleavage Assay

The activities of L-ribozymes and D-ribozymes were measured under different conditions. The basic conditions were as follows. 0.2 μM target RNA or DNA were mixed with 10 μl reaction mixture in the presence of 2 μM DNAzyme or RNAzyme in 50 mM tris-HCl buffer, pH 7.5, incubated at 20° C. for 2 hours (ratio DNAzymes or RNAzyme/target hence 10:1). Before the reaction, target RNA or DNA and DNAzyme or RNAzyme were denatured for 2 minutes at 72° C. and slowly cooled down to 25° C. (1° C/min.) in the heating block. The influence of Mg⁺⁺ ions in concentrations from 0.1 to 10 mM was investigated. Cleavage products were separated on 20% polyacrylamide gel electrophoresis in presence of 7M urea in 0.09 tris-borate buffer, pH 8.3. The analysis of the fluorescence was performed on Phosphoimager Fuji film FLA 5100. The data were obtained using the Fuji Analysis Program. Diagrams were created with Excel.

### EXAMPLE 2

### Preparation of the Target Sequences and the Ribozymes

The target sequences were prepared by way of chemical synthesis. The synthesis products had a purity of more than 90%.

As DNAzyme or RNAzyme sequences were selected, according to the target sequences, the variable regions of the DNAzyme or RNAzyme at the cutting site triplet, and the RNAzyme or DNAzyme sequences were synthetically prepared. The synthesis products had a purity of over 85%.

All synthesized products were marked with fluorescein at the 5′ end.

### EXAMPLE 3

### Measurement of Activities in Cells

HeLa cells were transfected with 1 μg EGFP plasmid according to instructions. Then followed an incubation with 25, 50 or 100 nM solution of the DNAzyme or RNAzyme to be used. After 24 h or 48 h, the cells were analyzed with a Leica microscope, or the fluorescence intensity (RFU) was measured according to instructions using the Multi-mode Microplate Reader Synergy-2.

### EXAMPLE 4

### Comparison of the Interaction of Different DNAzymes with Target Sequences

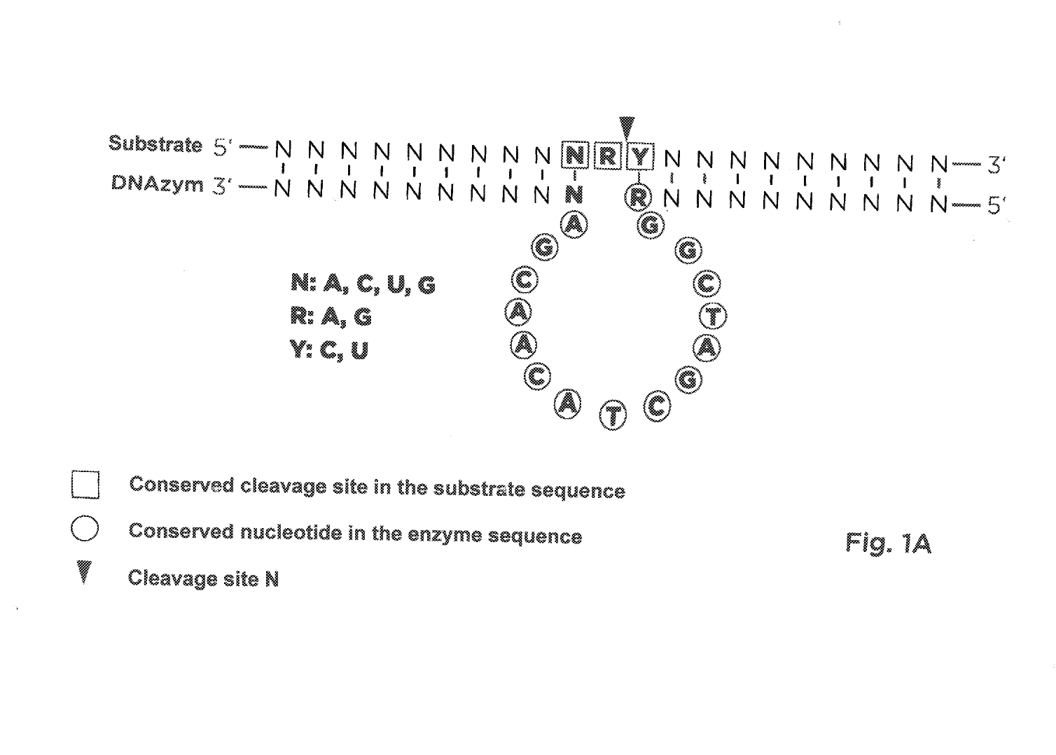

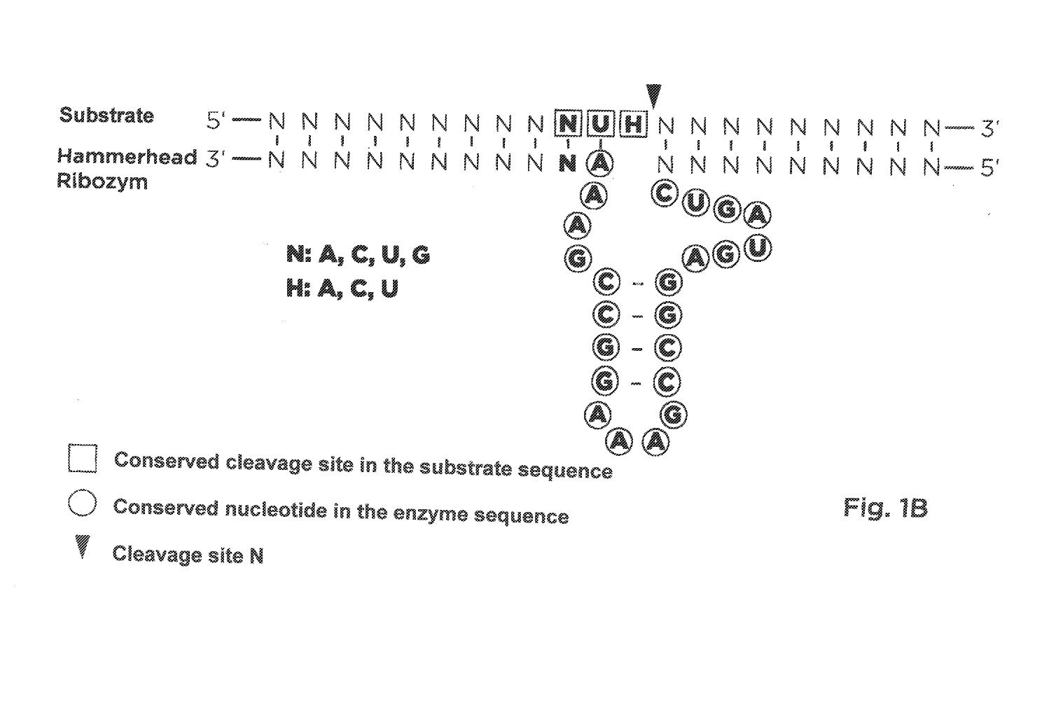

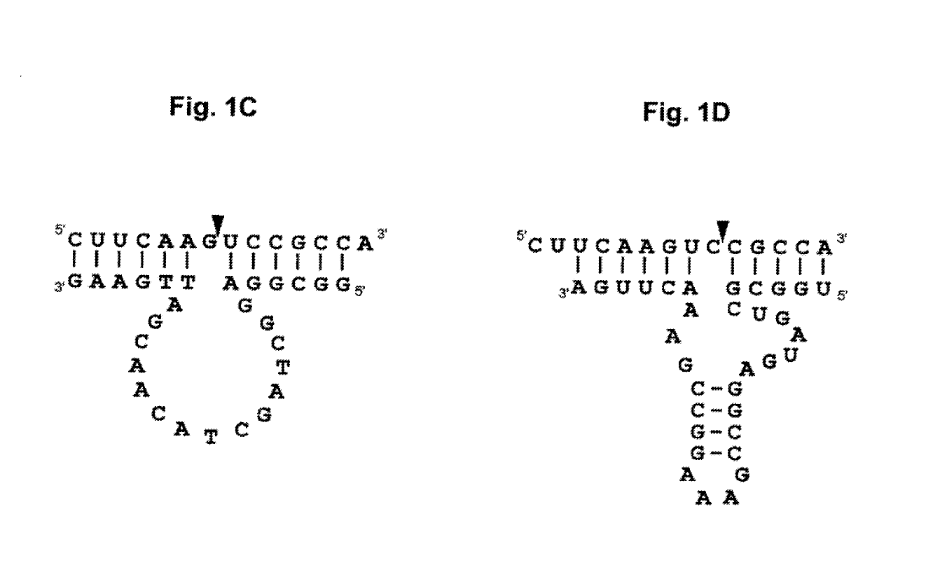

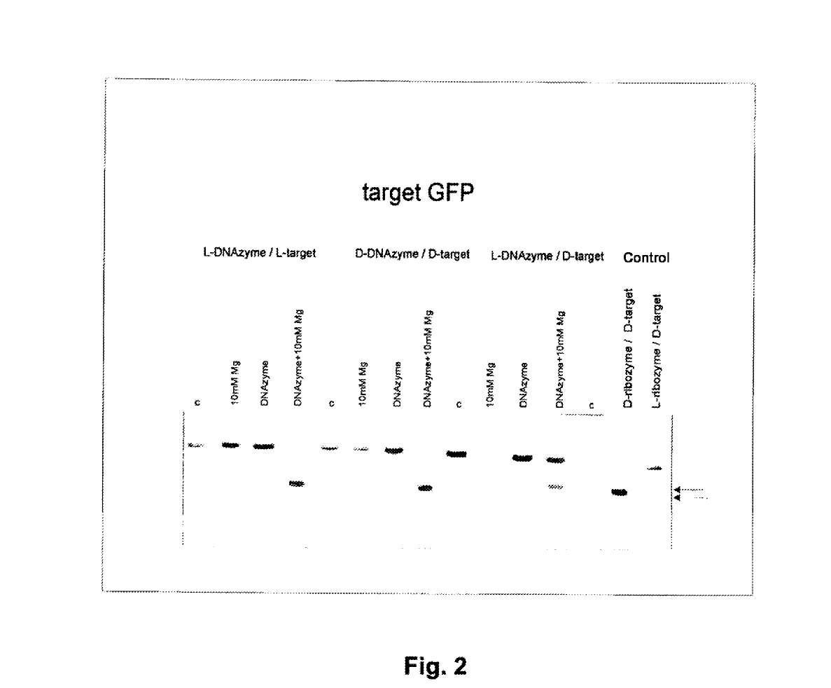

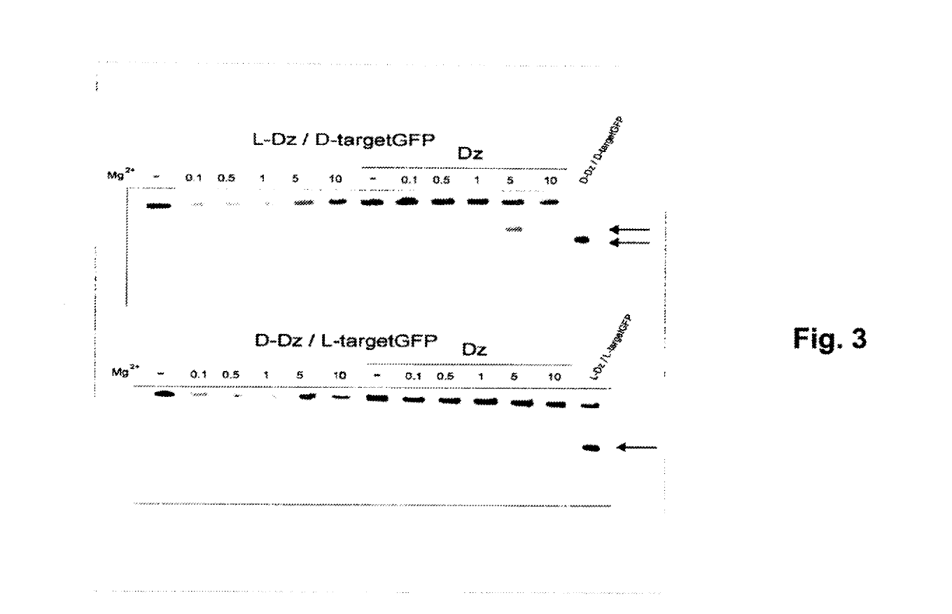

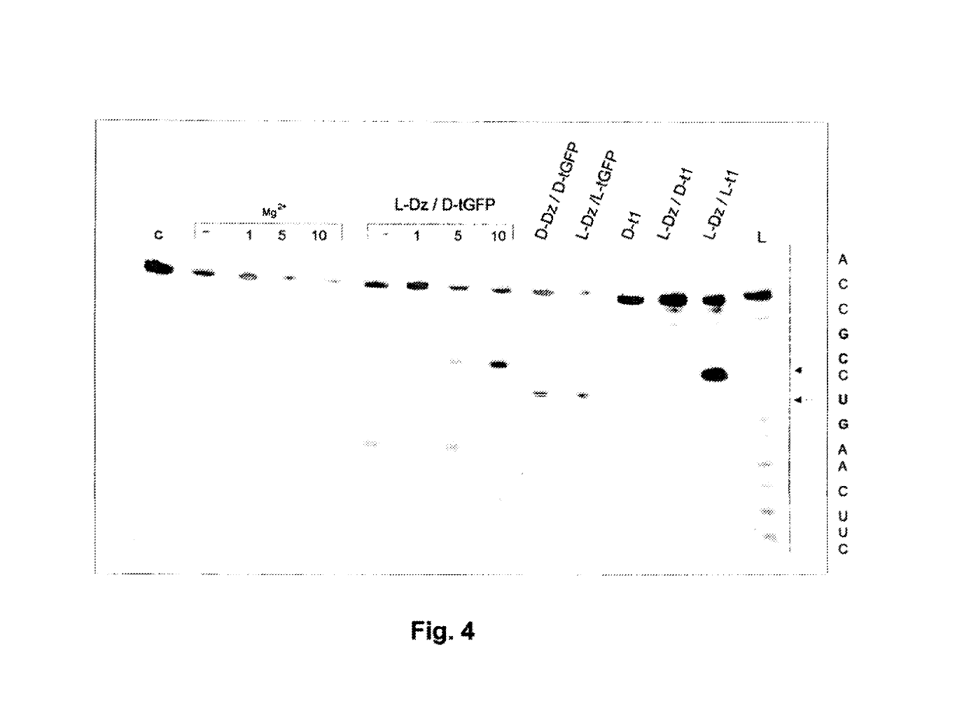

In FIG. 2 (10 mM MgCl₂), it can be seen that an L-DNAzyme is capable of cutting both the L-target sequence and the D-target sequence. FIG. 3 shows the dependencies on the MgCl₂ concentration. This figure also shows that L-DNAzyme cuts the D-target, but D-DNAzyme does not cut the L-target. FIG. 4 again shows measurements according to FIG. 3, in addition the cutting site at the target according to FIG. 1a being visible.

### EXAMPLE 5

### Activities of Different DNAzymes and RNAzymes in Cells

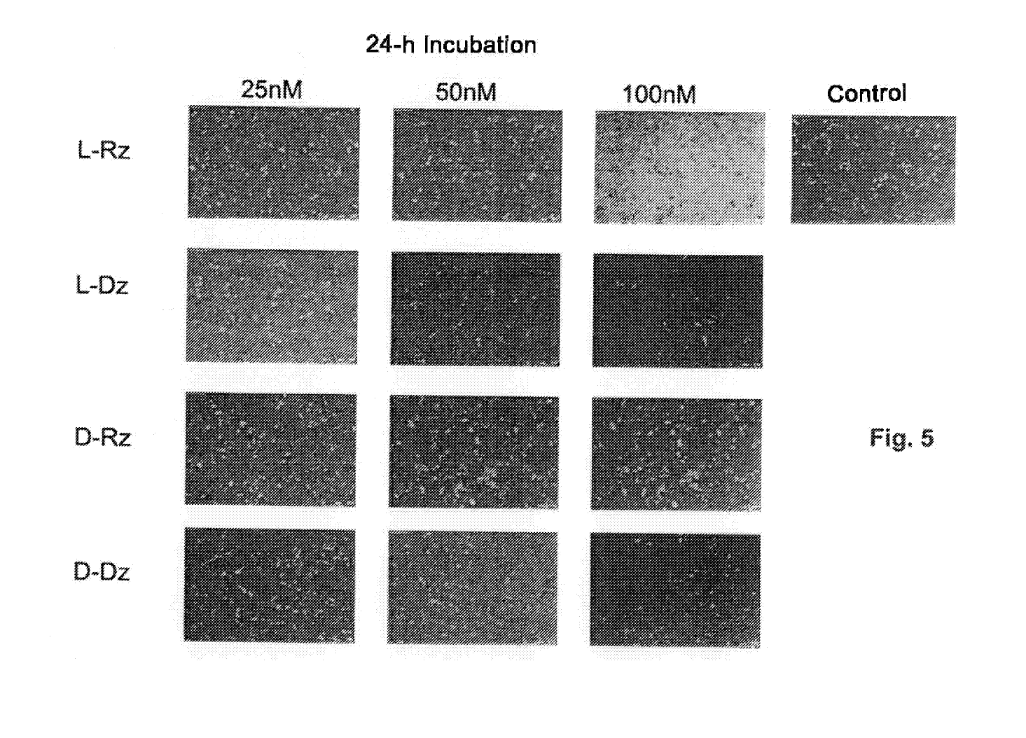

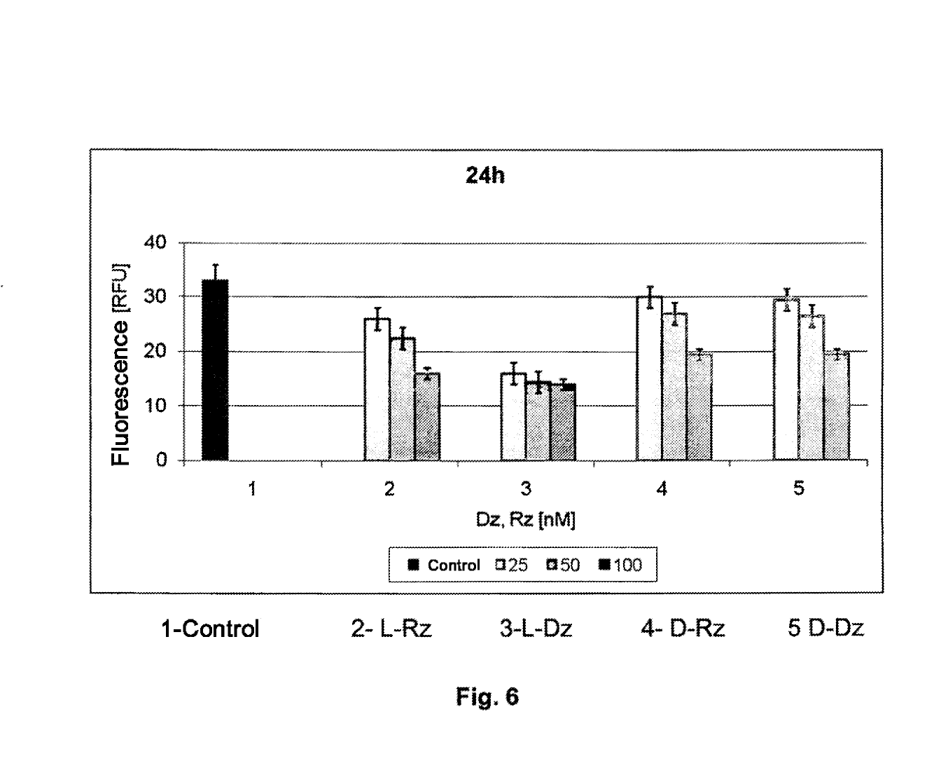

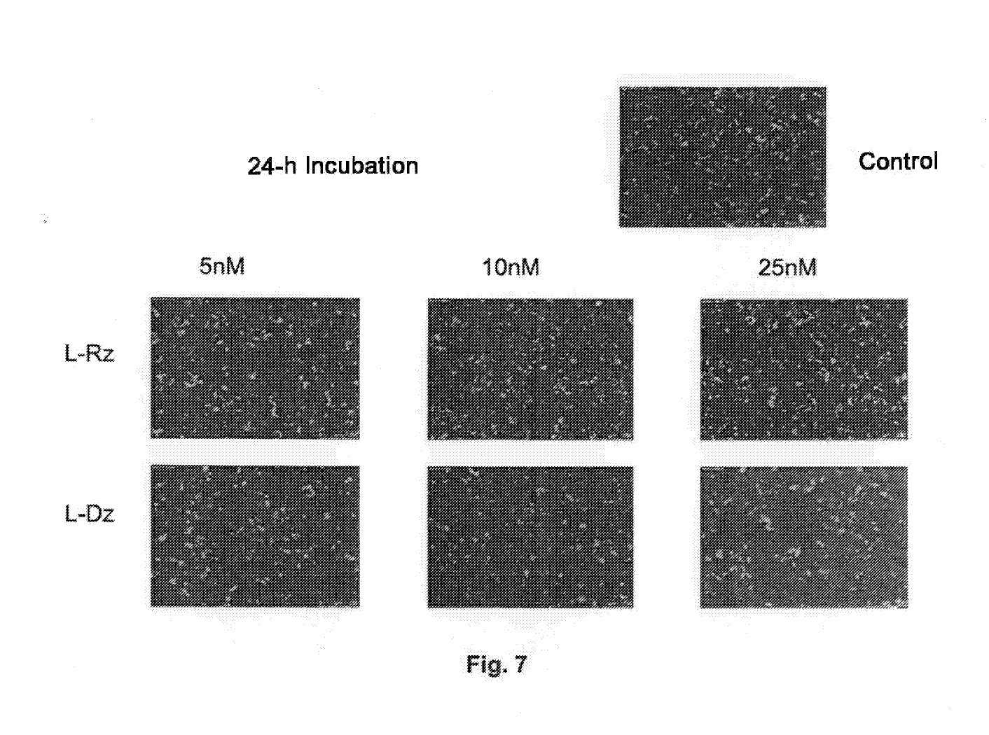

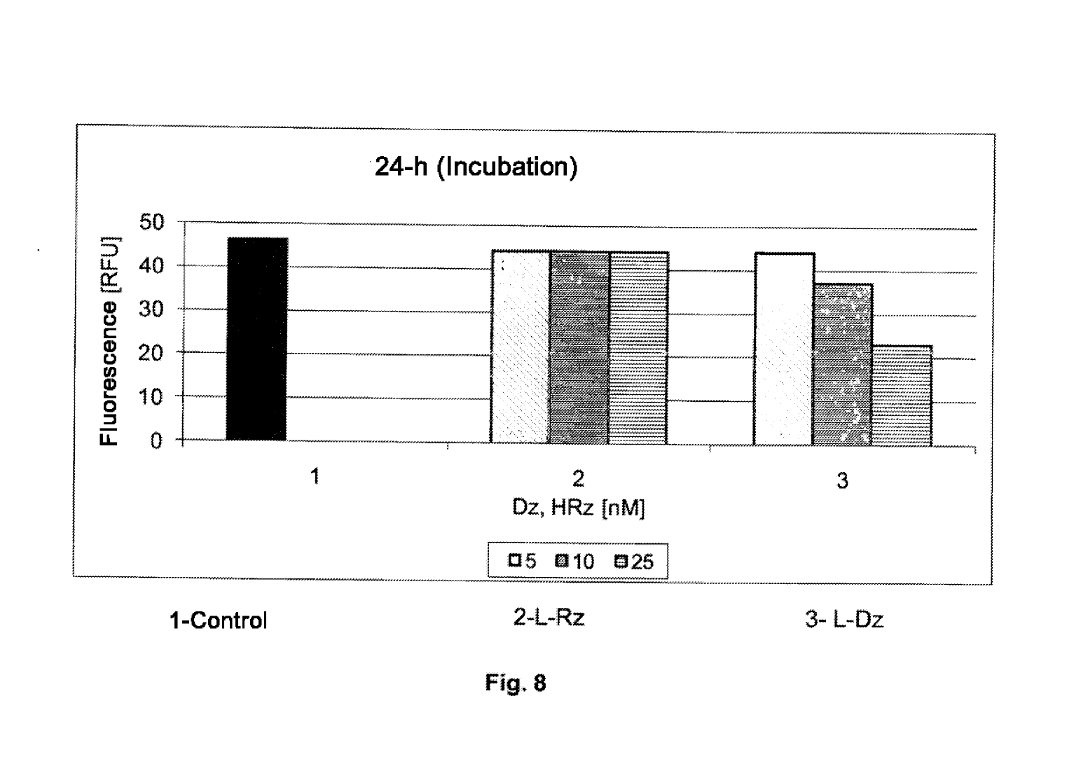

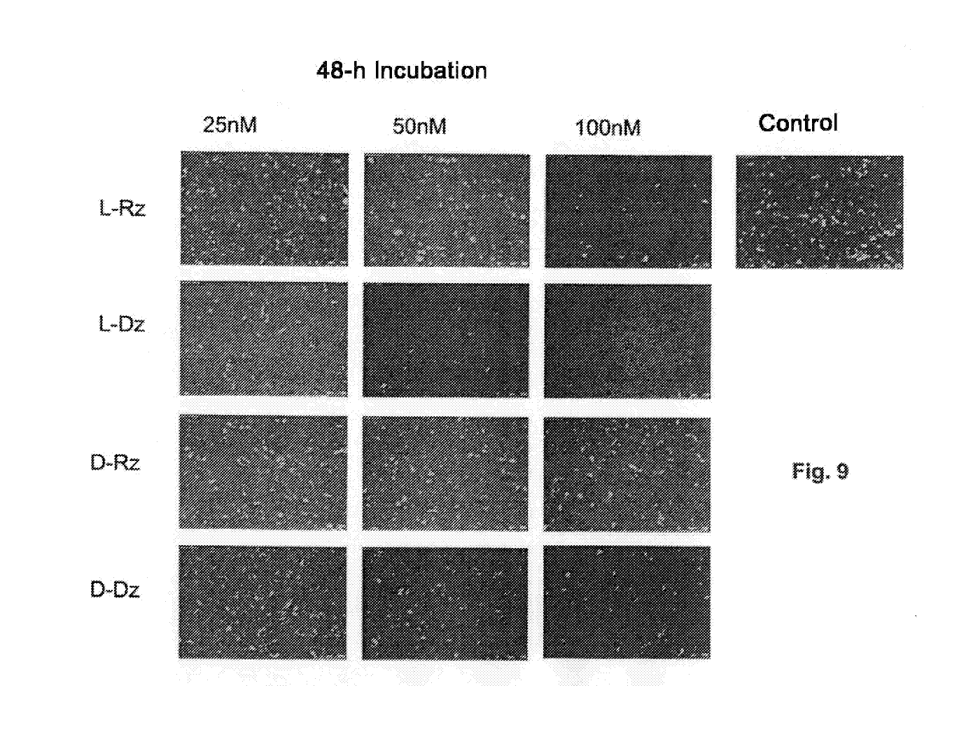

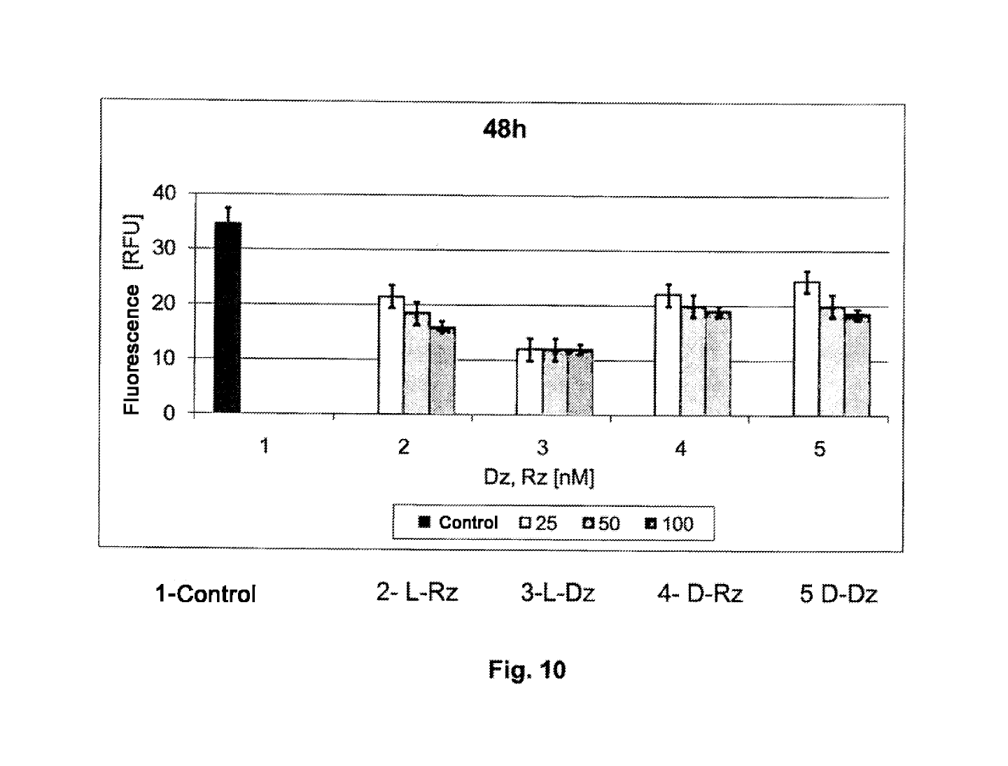

In FIG. 5, HeLa cells were transfected with EGFP plasmid, thus they contain a D-target. It can in particular be seen that L-DNA inhibits the fluorescence to a stronger degree than L-RNA, or also D-RAN or D-DNA. This finding is significantly confirmed in FIG. 6. Thereby, in particular the superior effect of L-DNA to L-RNA in the cell is proven. In FIGS. 7 and 8, these results are also confirmed for lower MgCl₂ concentrations. FIGS. 9 and 10, finally, show that L-DNA also shows a significantly better inhibition for 48 h incubation than the other nucleic acids.

### EXAMPLE 6

### Potential Pharmaceutical Applications

Besides the use as an antidote in the treatment with Spiegelmers, the invention can also be used in other general contexts. Thereto belong in principle all indications, where a disease is correlated with the undesired expression of a gene.

An example is heart failure and hypertrophy. From the document L. Suckau et al., Circulation 119: 1241-1252 (2009), it is known in the art that in this case a treatment is suitable, which inhibits phospholamban. In this document, RNAi is used for this purpose. Corresponding L-DNA can easily be constructed to human phospholamban based on the known sequence information, and the advantages of a better stability of the L-DNA ingredient against enzymatic degradation, compared to the in so far known treatment methods, will result, together with a good activity in terms of the inhibition of the target RNA coding for phospholamban.

Another target in the human organism, for example, is H19 RNA. This gene is differentially expressed, for example in cancer cells. Inter alia from the document US 2010/0086526 A, it is known in the art to inhibit H19 RNA by means of siRNA. In an analogous manner, an L-DNA molecule according to the invention can be selected that cuts known and suitable sites of H19 nucleic acids, so that the transcription thereof is reduced or inhibited. This results in advantages, as discussed above.

1. Use of an L-DNA for preparing a pharmaceutical composition.

2. The use according to claim 1, wherein the L-DNA is capable of binding to an L-RNA or D-RNA, in particular in an antisense reaction, and optionally cleaving the L-RNA or D-RNA in the range of a target sequence of the L-RNA or D-RNA.

3. The use of an L-DNA, which is capable of binding to an L-RNA or D-RNA, in particular in an antisense reaction, and optionally cleaving the L-RNA or D-RNA in the range of a target sequence of the L-RNA or D-RNA, for preparing a pharmaceutical composition for the treatment of undesired physiological side reactions due to the administration of a therapeutic molecule containing the L-RNA or D-RNA.

4. The use of an L-DNA for preparing a pharmaceutical composition for the treatment or prophylaxis of diseases, which are associated with an overexpression of at least one endogenous gene, the L-DNA being capable of binding to an endogenous target D-DNA or target D-RNA coding for the gene, in particular in an antisense reaction, and optionally cleaving a target sequence of the endogenous target D-DNA or target D-RNA coding for the gene.

5. The use according to claim 3, wherein the therapeutic molecule consists of the L-RNA, that is a double-stranded, for example a Spiegelmer.

6. The use according to claim 3, wherein the therapeutic molecule contains an aptamer covalently bonded with the L-RNA or antibodies covalently bonded with the L-RNA.

7. The use according to claim 3, wherein the pharmaceutical composition contains the L-DNA in at least the dose corresponding to the dose of administration of the L-RNA, preferably in a dose that corresponds to 2 to 100 times, preferably 2 to 20 times, the dose of administration of the L-RNA.

8. The use according to claim 3, wherein the pharmaceutical composition additionally contains a nucleic acid, in particular a 5 to 100-mer, which is capable of melting a double-stranded D-RNA or L-RNA in the range of the target sequence.

10. A pharmaceutical composition comprising an L-DNA for the treatment of undesired physiological side reactions due to the administration of a therapeutic molecule containing an L-RNA or a D-RNA.

11. A pharmaceutical composition comprising an L-DNA for the treatment or prophylaxis of diseases, which are associated with an overexpression of at least one endogenous gene, wherein the L-DNA is being capable of binding to an endogenous target D-RNA or target D-DNA coding for the gene, in particular in an antisense reaction, and optionally cleaving a target sequence of the endogenous target D-RNA or target D-DNA coding for the gene.

12. A method for preparing a pharmaceutical composition of claim 10 comprising a) creating and synthesizing a sequence and from L-deoxy-ribonucleotides, which is capable of binding to a predetermined sequence of L-ribonucleotides or a predetermined sequence of D-ribonucleotides or D-deoxyribonucleotides, b) is optionally capable of cleaving said predetermined sequence, and c) preparing the obtained L-DNA in a pharmacologically effective dose for administration.

13. The method according to claim 12, wherein the L-DNA is mixed with galenic auxiliary and/or carrier substances.
